(19) United States
(12) Patent Application Publication
(10) Pub. No.: US 2026/0232867 A1
(43) Pub. Date: Aug. 13, 2026

(54) HEOMSTATIC SEALANT POWDER FOR SEALING LEAKS IN TISSUES

(71) Applicants: GUANGZHOU BIOSEAL BIOTECH CO., LTD., Guangzhou Science City (CN); ETHICON, INC., SOMMERVILLE, NJ (US)

(72) Inventors: SHUANG CHEN, GUANGZHOU SCIENCE CITY (CN); YAPING WANG, GUANGZHOU SCIENCE CITY (CN); XUEQIN DENG, GUANGZHOU SCIENCE CITY (CN); YUFU LI, SOMMERVILLE, NJ (US); TINGWAN XIE, GUANGZHOU SCIENCE CITY (CN); HAI LI, GUANGZHOU SCIENCE CITY (CN); XUEMEI ZHANG, GUANGZHOU SCIENCE CITY (CN)

(21) Appl. No.: 19/156,953

(22) PCT Filed: Apr. 4, 2023
(86) PCT No.: PCT/CN2023/086100

§ 371 Date: Aug. 15, 2025

(51) Int. Cl. A61L 24/08; A61L 24/10

(52) U.S. Cl. CPC A61L 24/08; A61L 24/106; A61L 2300/418; A61L 2400/04

(57) ABSTRACT

Disclosed herein are powdered compositions comprised of fibrinogen, thrombin, glycerol-water solution, and optionally oxidized cellulose (OC), wherein the glycerol is present at a concentration ranging from about 0.7% to about 6%, by weight; and wherein the OC is present at a concentration below about 9%, by weight, or is absent. Methods of making the compositions, and uses of the compositions in methods for treating a bleeding tissue, and for sealing leaks in tissues are further disclosed.

### FIELD OF THE INVENTION

The present invention relates, inter alia, to hemostatic sealant powder comprising a blend of fibrinogen, and thrombin, e.g., for use for sealing leaks in tissues.

### BACKGROUND OF THE INVENTION

In various situations during surgery therapeutic agents are spread inside the human body, for prevention of bleeding after the surgery. Existing agents have drawbacks, for example, sheets are difficult to place in minimal invasive surgeries (MIS).

The selection of appropriate methods or products for the control of bleeding is dependent upon many factors, which include, but are not limited to, bleeding severity; anatomical location of the source and the proximity of adjacent critical structures, whether the bleeding is from a discrete source or from a broader surface area; visibility and precise identification of the source; and access to the source.

In an effort to address the above-described problems, materials have been developed for controlling excessive bleeding. Topical Absorbable Hemostats (TAHs) are widely used in surgical applications. TAHs encompass products based on oxidized cellulose (OC), gelatin, collagen, chitin, chitosan, etc. To improve the hemostatic performance, scaffolds based on the above materials can be combined with biologically-derived clotting factors, such as thrombin and fibrinogen.

Fibrin sealants, also known as fibrin glue, have been in use in the clinic for decades. Oftentimes, fibrin sealant consists of two liquid components, a fibrinogen comprising component and a thrombin comprising component, which are stored frozen due to their inherent instability. Sometimes fibrin sealant products consist of two freeze dried components, which require reconstitution immediately prior to use and delivery by a conjoined syringe or other double barreled delivery device.

Dry sealant powder, when mixed with fluid, can form a paste or slurry that is useful as a flowable, extrudable and injectable hemostat and is made of plasma-derived components: e.g., (a) fibrinogen mixture, primarily composed of fibrinogen optionally along with catalytic amounts of Factor XIII and (b) a high potency thrombin. Due to its biodegradability, and its bactericidal and hemostatic properties, OC based materials such as oxidized regenerated cellulose (ORC), have long been used as topical hemostats. OC- and ORC-based materials are also used as an adhesion barrier. Products based on ORC are used in a variety of surgical procedures, including: neurosurgery, abdominal surgery, cardiovascular surgery, thoracic surgery, head and neck surgery, pelvic surgery and skin and subcutaneous tissue procedures. Several methods for forming various types of hemostats based on OC materials are known, whether made in powder, woven, non-woven, knit, and other forms. Currently utilized hemostats include powder, or fabrics comprising ORC.

The effect of fibrin sealant in sealing defects in tissues which are under constant movement, e.g., gastrointestinal tissue and lung tissue, has been reported. However, there is discrepancy in these reports. One approach to diminish post-operative defects such as leaks at the staple or suture (staple/suture) line after e.g., gastrointestinal resection is to use staple/suture line reinforcements. Different staple/suture line reinforcement products are commercially available. Other approaches use fibrin as absorbable staple line reinforcement for the prevention of post-operation leaks.

US Patent No. U.S. Ser. No. 10/111,980B2 discloses is a dry composition comprising one or more polyols, which upon addition of an aqueous medium forms a substantially homogenous paste suitable for use in haemostasis procedures. The paste reconstitutes spontaneously upon addition of the liquid.

U.S. patent publication No. US20210060204A1 describes a biocompatible hemostatic composition and a biocompatible tissue sealant, which when used in combination provides a safe and effective method of achieving hemostasis. The biocompatible composition and sealant may be applied either on a surface of the patient's body, or inside the body cavity.

PCT publication No. WO2021128050A1 discloses a non-flowable and deformable hemostatic compositions comprised of a xerogel crosslinked powdered polysaccharide dispersed within a substantially anhydrous blend of: glycerol, and polyethylene glycol (PEG), wherein the PEG and glycerol are present in a ratio ranging from higher than 1:1.3: to below 1:2.7 by weight, respectively, and wherein the powder content in the composition is above 50%, by weight.

U.S. patent Ser. No. 11/413,335 discloses a method of making the hemostatic materials a non-aqueous solvent in a High Shear Mixing (HSM) apparatus, and performing high shear mixing and regular mixing and/or agitation, while allowing the volatile non-aqueous solvent to evaporate through any suitable port(s) such as through air seals of mixing blades/impellers and/or through a vacuum tube.

### SUMMARY OF THE INVENTION

The present invention relates, inter alia, to a powdered blend of fibrinogen, thrombin, and a small amount of glycerol-water solution.

An object of the present invention is to provide compositions of dry powder that is capable of being fast dissolved in aqueous medium to form gel layer with significantly increasing gel strength and adhesive strength. The sealant powder has both hemostatic and sealing indications and is produced by a novel scale-up granulation process.

The granulation process of making the powder includes leveraging the reaction of the biological materials themselves (fibrinogen and thrombin) with water to form a small amount of fibrin gel as the binding agent without the need to introduce new polymer binder, thereby reducing the concerns of safety and biocompatibility. Briefly, the novel high shear mixer granulation process in the present invention includes: raw materials of biological components (dry powder), then mixing, then spraying certain amount of water for injection (WFI), then mixing with certain ratio of glycerol, and then granulation. The use of glycerol (instead of a low-boiling point solvent) is also advantageous since low-boiling point solvents might be hard to reduce to a safety level from a final product.

The composition may easily be applied to a site of need, including in difficult-to-reach areas of the body. The disclosed fibrin sealant powder with glycerol has a better gel strength, can enhance adhesive strength e.g., for Canine model, with the possibility to decrease ORC amount, and the solubility appears to be similar to, or better than, with the absence of ORC. The water content for fibrin sealant powder with glycerol can be below 2.5%, which has significant impact on the powder stability.

Accordingly, the present invention is based, inter alia, on the surprising finding that micro aggregate composition comprising fibrinogen, thrombin, glycerol, fibrin at certain respective amounts, and optionally ORC, produced by a novel a granulation process (e.g., High Shear Mixing (HSM)) can fast dissolve to form gel layer to seal tissue and stop bleeding, as well as forming a gel with sufficient strength and adhesive forces which may be uses for sealing leakage from injured tissue without tamponade.

The formed powder is therefore ready-to-use and can be directly sprayed onto the target site.

According to an aspect of the present disclosure, there is provided a powdered composition comprising fibrinogen, thrombin, glycerol-water solution and optionally oxidized cellulose (OC), wherein: (i) the glycerol is present at a concentration ranging from about 0.7% to about 6.5%, by weight; (ii) the water is present at a concentration of less than 3%, by weight, and wherein: (iii) the OC is present at a concentration below about 9%, by weight, or is absent.

In some embodiments, the composition further comprises fibrin.

In some embodiments, the OC comprises oxidized regenerated cellulose (ORC).

In some embodiments, the composition is free of OC and/or ORC.

In some embodiments, the composition is free of non-aqueous low-boiling solvent.

In some embodiments, the composition is free of hydrofluoroether.

In some embodiments, the composition is a fast soluble solid form in an aqueous medium, e.g., being dissolved within less than 3 min at 25° C.

In some embodiments, the powder comprises particles characterized by a size distribution of D90 being at most 500 μm.

In some embodiments, the composition is ready-for-use for hemostatic and/or sealing indication.

In some embodiments, the composition by characterized by forming a gel in an aqueous medium, the gel having a high gel strength ranging from e.g., 85 kPa to 160 kPa.

In some embodiments, the composition is devoid of additional polymeric binder other than fibrin.

In some embodiments, the composition is for use in a method for treating a bleeding tissue, and/or preventing leak, e.g., air leak, in an organ in a need thereof, e.g., in minimal invasive surgeries.

In some embodiments, the composition further comprises a calcium salt.

In some embodiments, the composition is deposited on an article (e.g., a patch).

According to an aspect of the present disclosure, there is provided a method of making a powdered hemostatic composition, comprising the steps of: (a) mixing a solid fibrinogen, thrombin, and optionally ORC fibers, forming a mixture; (b) inserting the mixture in high shear mixing reactor; (c) adding a solution of glycerol and water to the mixture in the reactor; (d) drying and sieving the composition obtained in step (c); and thus forming the powdered hemostatic composition.

In some embodiments, the glycerol is present in the solution at a concentration ranging from more than about 10% to less than about 40%, by weight.

In some embodiments, the water is added in a quantity preventing full clotting of fibrinogen.

In some embodiments, the mixture is substantially free of OC and/or ORC.

According to an aspect of the present disclosure, there is provided a powder composition obtainable by the method disclosed herein in any embodiment thereof.

According to an aspect of the present disclosure, there is provided composition comprising a mixture of solid fibrinogen, thrombin, and optionally ORC fibers in solution of water and glycerol, wherein the glycerol is present in the solution at a concentration ranging from more than about 10% to less than about 40% by weight. In some embodiments, the composition further comprises fibrin.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1B present images—a photographic image (FIG. 1A) and a scanning electron microscopic (SEM) image (FIG. 1B) of an aggregated powder prepared according to an embodiment of the present disclosure (Sample 15-02, see Table 6).

FIGS. 2A-2B present graphs showing the tensile stress (FIG. 2A) and the tensile strain (FIG. 2B) of a fibrin gel made from the aggregated powder prepared according to embodiments of the present disclosure compared to gels made from powders produced by hydrofluoroether (HFE) (based on Tables 4 and 5, respectively).

FIGS. 3A-3B present graphs showing present graphs showing the tensile stress (FIG. 3A) and the tensile strain (FIG. 3B) of a fibrin gel made from the aggregated powder prepared according to embodiments of the present disclosure (based on Table 6).

FIG. 4 presents photographic images showing the results of solubilization testing of the comparative compositions prepared using various amount of glycerol in a water-glycerol dispersant: upper panel—10% glycerol (v/v %); middle panel—20% glycerol (v/v %); lower panel—40% glycerol (v/v %). Each panel of glycerol volume is divided to various amount of oxidized regenerated cellulose (ORC)—“10:1” means that every 100 g biological components match with 10 g of ORC component; “20:1” means that every 100 g biological components match with 5 g of ORC component; and “0” means no ORC added in the formulation (from upper to lower in each panel of glycerol volume). The solubility was tested at 0 min, 1 min, 2 min, and 3 min (from left to right in each panel of glycerol volume); see also Table 7.

FIG. 5 presents graphs showing the comparative results of solubilization testing of the comparative compositions based on FIG. 4 and Table 7 below.

FIG. 6 presents graphs showing the water content % (by weight) of powders made according to some embodiments as measured by Carl Fischer's Titration method (based on Table 8).

FIG. 7 presents a photographic image illustrating a relatively poor solubility of an HFE granulated sample.

FIGS. 8A-8B present photographic images demonstrating adhesive properties of the glycerol granulated powder to the porcine lung tissue in this ex vivo test (FIGS. 8A —before applying the powder, FIGS. 8B—after applying the powder and obtaining sealing).

FIGS. 9A-9D present photographic images demonstrating steps 1, 2, 4, and 5, respectively, of the Liver Resection Model as described in the Examples section.

### DESCRIPTION OF EMBODIMENTS OF THE INVENTION

Commercial preparations of thrombin and fibrinogen blend exist as a powder. Application of powder for a limited and specific site can be difficult to achieve. An object of the present invention is to provide a powder composition that is capable of being fast dissolved in aqueous medium to form gel layer with significantly increased gel strength (also referred to as “tensile strength”) and can address hemostasis and tissue leak at the same time.

The disclosed powder composition is characterized by components being uniformly distributed within.

According to an aspect of the present disclosure, there is provided a powdered composition comprising fibrinogen, thrombin, glycerol-water solution and optionally oxidized cellulose (OC), wherein: (i) the glycerol is present at a concentration ranging from about 0.7% to about 6.5%, by weight; and wherein: (ii) the OC is present at a concentration below about 8.50%, by weight, or, in some embodiments, is absent.

The presence of water in such a composition is counter-intuitive, and it may be assumed, without being bound by any particular theory or mechanism, that after mixing the powder in the dispersant the uniformly dispersed biologics forms a fibrin in a glycerol/water system. Upon application in a bleeding site the fibrin matrix serves as a binder so the powder components are homogeneously distributed.

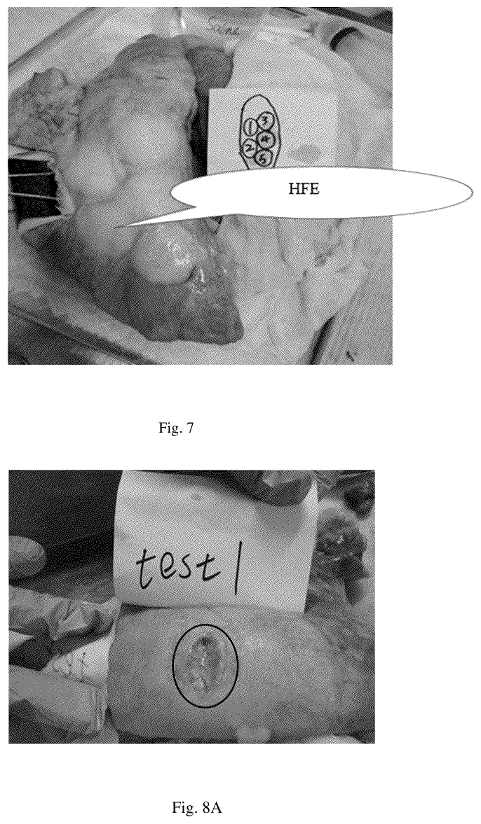

The presence of water is low as it added in a quantity preventing full clotting of fibrinogen. That is, in some embodiments, the amount of water in the powder is less than 3%, by weight. In some embodiments, the amount of water in the powder is less than 2.5%, by weight. In some embodiments, the amount of water in the powder is less than 2%, by weight. In some embodiments, the amount of water in the powder ranges from about 1.5% to about 2.5%, by weight (see e.g., FIG. 6).

Thus, in some embodiments, the composition is homogeneous. As used herein, by “homogeneous” it is meant to refer to a uniform composition and texture throughout, that is, the powder and the small amount of the dispersant are dispersed substantially evenly therethroughout. Herein, “dispersed substantially evenly” is meant that the differences of the concentration of the components in the powder varies within less than ±30%, less than ±20%, less than ±10%, less than ±5%, by weight, therethroughout, that is, e.g., without a significant solid-liquid phase separation. Typically, term “homogeneous” refers to a uniform appearing product which withstands a liquid/solid phase separation (e.g., less than 4% by weight) at 8 to 40° C., 10 to 30° C., or 20 to 30° C.

The term “powder” or “powdered composition” as used herein, generally refers to a solid having fine particles. Powders may be granular materials.

In some embodiments, the powder is in the form of aggregate(s) or granulate(s).

The term “aggregate” describes a particle formed from assembled components. Aggregates may optionally be made by a step of humidifying the powder composition; compacting, e.g., by roller and/or slugging the powder to form aggregates; dehumidifying; milling; sieving the aggregates; and optionally dosing the resulting aggregates into a storage container or into a delivery device. The term “granulate” or “granulate material” may particularly denote a conglomeration of discrete solid particles typically below 550, below 500 micron, below 400 micron, or below 350 micron. The aggregate is also referred to as “micro-aggregate”.

In exemplary embodiments, high shear mix is utilized to assemble particles of fibrinogen, thrombin, and optionally CaCl₂, a buffering agent such as lysine, albumin (that is present in the source from which the fibrinogen is collected), and/or ORC into aggregates. In another embodiment, the composition is in the form of a mixture. The mixture may comprise, in exemplary embodiments, spray-dried first microparticles that comprise fibrinogen, combined with spray-dried second microparticles that comprise thrombin, e.g., porcine thrombin, together forming particles.

The term “solid” characterizes the state of the compound or composition at room temperature (e.g., 25° C.) and at atmospheric pressure (760 mmHg), i.e. a compound or a composition of high consistency which retains its form during storage. This term in the present application also relates to non-fluid particles, or dissolved substance. As opposed to “liquid” compounds and compositions, the solid does not flow under its own weight.

In some embodiments, the powder is spray-dried and/or high sheared mixed.

Embodiments of spray drying and high-shear mixing are described hereinthroughout.

The term “spray drying” is used herein in a broad sense, which include, without being limited thereto, processes for transforming a solid dissolved or suspended in a liquid into a powdery. Typically, spray drying is a drying method used to create powder from a solution, suspension or emulsion that is atomized through a spray nozzle in a hot airflow and dried instantly. Spray drying process for producing thrombin and fibrinogen powders, which maintain their activity may be controlled by several process parameters, among them are, without limitation: column air flow and temperatures, nozzle size and atomizing air flow rate, and material flow rate. Non-limiting exemplary parameters used in order to establish a robust process to produce a blend of thrombin and fibrinogen (e.g., in a powder form) which maintains its activity are provided in the Examples section below.

The term “high-shear mixing” generally means a turbulent-flow type of mixing. In some embodiments, the high-shear mixing is characterized by speed of mixing blade and/or speed of cutting blade ranging from: 150 to 500 rpm.

Additionally, or alternatively, other methods may be used for obtaining dry composition, such as, without limitation, lyophilization of the fibrinogen solution and/or thrombin solution, followed by micronization. Typically, following lyophilization of the solution, a porous and spongy solid material, referred to as a “cake” (may also be referred to herein as “solid composition”) is obtained.

The term “micronization” is used when the particles that are produced e.g., from the solid composition, are only a few micrometers in diameter. Micronization can be achieved by processes including, but not limited to, jet milling, pearl-ball milling, high-pressure homogenization, the RESS (Rapid Expansion of Supercritical Solutions) process, the SAS (Supercritical Anti-Solvent) method, or the PGSS (Particles from Gas Saturated Solutions) method. Typically, but not exclusively, micronization results in a 30 to 400-fold size reduction of the protein powder from its original size.

In some embodiments, the composition is stable.

The terms “stable”, and “stability” when referring to the disclosed powder, mean that an active component within, e.g., thrombin at a certain temperature and after certain time duration remains at least 70% active, that is, capable of forming a fibrin clot.

The powder can be roller compacted or slugging compacted and then may be subjected to pre-breaking, dehumidification, and subsequently followed by a step of final milling. The resulting aggregates may be selected to a targeted hemostatic aggregate fraction, e.g., by sieving for desired size and/or distribution separation (e.g., below 500 μm, or below 250 μm, in a defined range of D50 and D90).

The terms “D50”, and “D90” refers to 50% and 90%, respectively (by numbers or volume), of the powder having a size that is less than or equal to the corresponding value.

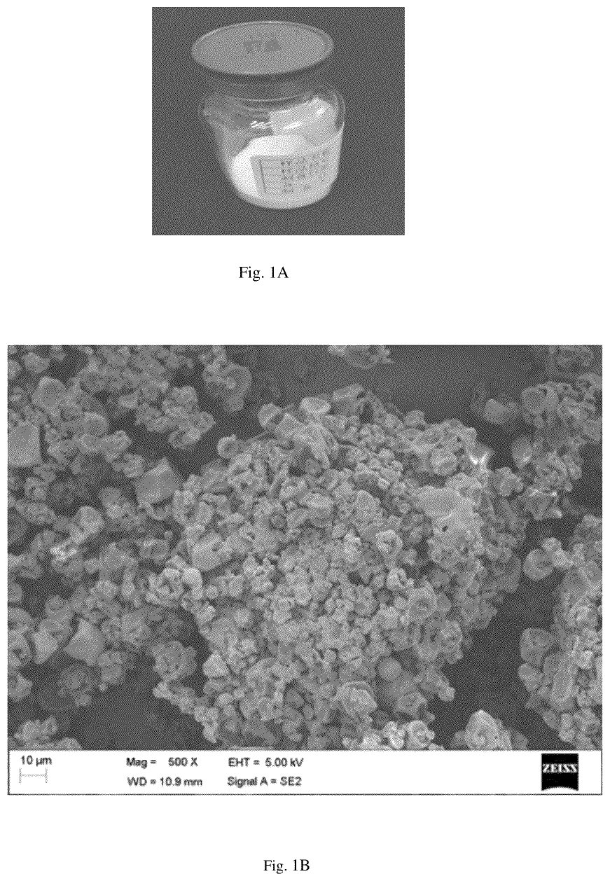

In some embodiments, the aggregate is characterized by a size distribution of D90 of less than 500 μm, and D50 of less than 180 μm. In some embodiments, the aggregate is characterized by D50 ranging from about 150 μm to about 180 μm. Reference is made to FIG. 1B presenting a SEM image of a powder produced according embodiments of the present disclosure.

The term “glycerol” means 1,2,3-propanetriol, and is also known as “glycerin”, and may also meant to also encompass a derivative thereof. The term “derivative” refers to compound containing essential elements of the parent substance.

In some embodiments, the glycerol is present at a concentration ranging from about 0.7% to about 6.5%, by weight. In some embodiments, the glycerol is present at a concentration ranging from about 1% to about 6%, by weight. In some embodiments, the glycerol is present at a concentration of about 0.5%, 1%, 1.5%, 2%, 2.5%, 3%, 3.5%, 4%, 4.5%, 5%, 5.5%, 6%, or 6.5%, by weight, including any value and range therebetween.

As used herein, “thrombin” denotes an activated enzyme which results from the proteolytic cleavage of prothrombin (factor II). Thrombin may be produced by a variety of methods of production known in the art, and includes, but is not limited to, recombinant thrombin and plasma derived thrombin. Human thrombin is a 295 amino acid protein composed of two polypeptide chains joined by a disulfide bond. Both human and non-human (e.g., bovine and porcine) thrombin may be used within the scope of the present disclosure.

The origin for thrombin used in this invention may be from one or several sources including but not limited to: plasma (e.g., porcine plasma), recombinant bacteria and/or cells (Vu et al., 2016, J. Viet. Env. 8(1):21-25), whole blood (pooled from several donations or not) and/or blood fraction (that may be pooled from several donations, e.g., plasma). Thrombin is available by manufacturers such as Johnson and Johnson, Baxter and CSL Behring either as a standalone product, e.g., EVITHROM®, or as a component of a product e.g., EVICEL®, TISEEL®, Beriplast®, and the like.

In some embodiments, the thrombin is present at a concentration of 3% to 10% or 4% to 5%, by weight of the powder. In some embodiments, the thrombin is present at a concentration of 3%, 3.5%, 4%, 4.5%, 5%, 5.5%, 6%, 6.5 or 7%, by weight of the powder, including any value and range therebetween. The activity may range from 100 to 1500, or, in some embodiments, from about to about 300 to 1200 IU per dose thrombin powder.

The term “fibrinogen” without more is intended to include any type of fibrinogen. “Fibrinogen” refers to monomeric and dimeric fibrinogen molecules having the monomer structure (AαBβγ), hybrid molecules, and variants thereof, whether naturally occurring, modified, or synthetic. The term “fibrinogen” may refer to fibrinogen from humans, but may include fibrinogen of any species, especially mammalian species, such as fibrinogen produced from porcine plasma.

Hereinthroughout, the combination of thrombin, fibrinogen and amino acids e.g., lysine, if present in the composition or the powder, is also referred to as “the biological parts”.

In some embodiments, the fibrinogen is present at a concentration of 60% to 95%, or 70% to 90%, by weight of the blend. In some embodiments, the thrombin is present at a concentration of 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95%, by weight of the blend, including any value and range therebetween.

The term “oxidized cellulose” (or “OC”) refers to a cellulose derivative in which at least some of the primary alcohol groups, e.g., on the carbon 6 of the anhydroglucose unit is oxidized to a carboxylic acid, and is optionally functionalized. OC may include materials, products, articles, or compositions comprising or consisting essentially of OC, e.g., a dressing, fibrin glue, synthetic glue, pad, matrix, powder, tab, pill, suture, fiber, stent, implant, scaffold, solution, gel, wax, gelatin, and the like.

In some embodiments, the OC (e.g., ORC) is in the form of a plurality of fibers. In some embodiments, the OC (e.g., ORC) is in the form of a plurality of fibers having a median size below 80 μm. In some embodiments, the OC (e.g., ORC) is in the form of a plurality of fibers having a median size ranging from 50 to 100 μm, or 60 to 80 μm, e.g., 50, 60, 70, 80, 90, 100 μm, including any value and range therebetween.

OC may be produced by applying an oxidizing agent on cellulose. The oxidizing agent may be selected from, without being limited thereto, chlorine, hydrogen peroxide, peracetic acid, chlorine dioxide, nitrogen dioxide, persulfates, permanganate, dichromate-sulfuric acid, hypochlorous acid, hypohalites, periodates, or any combination thereof, and/or a variety of metal catalysts. Oxidized cellulose may contain carboxylic acid, aldehyde, and/or ketone groups, instead of, or in addition to, the original hydroxyl groups of the starting material, cellulose, depending on the nature of the oxidant and reaction conditions.

The term “contact”, or any grammatical variant thereof, is used in its broadest sense and refers, for example, to any type of combining action which brings e.g., the hemostatic composition into sufficiently close proximity with water, aqueous solution, or blood, such that a clot or gel can be formed.

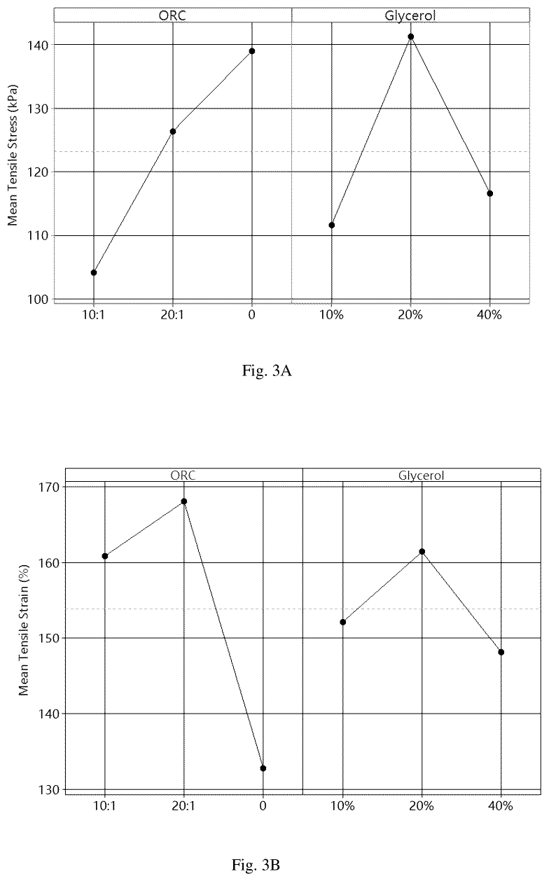

In some embodiments, the composition is substantially devoid of OC or ORC, as it also appears that the addition of ORC might decrease the gel strength property (see e.g., Table 6 in the Examples section that follows and FIG. 3A).

In some embodiments, the water absorbing agent (e.g., ORC), if not absent, is present at a concentration of up to 3%, up to 8%, up to 10%, up to 15%, up to 20% or up to 25%, e.g., in the range of 3 to 15%, by weight of the blend (i.e. powdered composition). In some embodiments, the water absorbing agent is present at a concentration of about 9% by weight of the powder.

In some embodiments of any aspect of the composition disclosed herein, the powder may comprise an additive e.g., calcium salt and/or one or more excipients, e.g., selected from, without being limited thereto, one or more amino acids, albumin, saccharides, and/or saccharide derivatives.

The term “additive” is meant to be understood as any substance that can be added or originates from a source material, to a composition, and may also include an active additive such as calcium salt as described below.

The term “excipient” as used herein denotes a non-active or non-therapeutic agent added to a pharmaceutical composition e.g., to provide a desired consistency or stabilizing effect.

Calcium is an important element in the clotting cascade, and may be needed for activation of factor XIII into factor XIIIa, which cross-links and stabilizes fibrin to generate an insoluble clot.

Accordingly, in some embodiments of any aspect of the disclosed composition, the blend further comprises additive such as, without limitation, calcium. Calcium used with the invention may be in the form of a salt, e.g., calcium chloride salt. Alternatively, additional salts may be used, such as calcium acetate and/or calcium citrate.

In some aspects, the disclosed composition, in any embodiment thereof, is provided in a kit.

In a kit, the calcium salt may be provided in the composition comprising the blend. Alternatively, the excipient(s), and/or the calcium salt, may be provided in a kit in a separate container, or the excipient(s), and/or the calcium salt, may be provided in a kit in the same container comprising the blend component.

In some embodiments of the kit or the composition, the calcium salt, e.g., calcium chloride (CaCl₂) is present at a concentration of 0.5% to 4%, or 2.5% to 3.5%, by weight of the blend. In some embodiments, the calcium salt, e.g., calcium chloride is present at a concentration of 0.5%, 1%, 1.5%, 2%, 2.5%, 3%, 3.5%, or 4%, by weight of the blend, including any value and range therebetween.

It is known that OC is acidic, and this acidity causes plasma proteins such as thrombin and fibrinogen to denature immediately; therefore, the co-administration of oxidized cellulose and thrombin and/or fibrinogen may lead to the plasma proteins being rendered ineffective.

In some embodiments, a buffering agent is added to the composition for pH adjustment. The buffering agent allows pH neutralization and provides an increased adhesiveness and sealing properties as compared to a control composition comprising the same aggregate having the components without comprising the buffering agent.

In some such embodiments, a buffering agent e.g., Tris, or Tris(hydroxymethyl)aminomethane buffer, or lysin is added in a powder form to the fibrinogen, thrombin, and ORC blend/mixture/powder for pH adjustment (e.g., to pH of about 7). In some embodiments, the buffering agent e.g., Tris or lysine, is present at a concentration of, 0 to 25%, 1% to 8%, or 1% to 5%, by weight of the blend/mixture/powder. In some embodiments, the buffering agent e.g., Tris or lysine, is present at a concentration (%) of 1, 2, 3, 4, 5, 6, 7, 8, 9, 10, 11, 12, 13, 14, 15, 16, 17, 18, 19, 20, 21, 22, 23, 24, or about 25%, by weight, including any value and range therebetween.

In some embodiments of any aspect of the compositions, the blend further comprises an excipient selected from amino acids e.g., lysine. In some embodiments of any aspect of the kit and/or compositions, the blend comprises OC and lysine.

In some embodiments, the composition is free of non-aqueous low-boiling solvent. The term “non-aqueous low-boiling solvent” refers to an organic solvent having a boiling point of not more than 150° C. at normal pressure. In exemplary embodiments, the composition is substantially free of hydrofluoroether (HFE).

In some embodiments, the composition further comprises fibrin.

The term “fibrin” does not only refer to fully coagulated fibrinogen but further includes any mixture of fibrin and fibrinogen which may occur during formation of fibrin from fibrinogen using thrombin and, thus, includes any ratio of fibrinogen/fibrin and any grade of gelation and/or clotting conceivable as long as it has no negative impact on the final pasty texture of the composition. Moreover, the term “fibrin” further includes any partly or fully cross-linked, gelled or clotted form.

It is thus to be appreciated that fibrin per se exists as a polymeric, insoluble matrix comprising fibrils, each fibril comprising many fibrin molecules.

In some embodiments, the composition is devoid of additional polymeric binder other than fibrin.

In some embodiments, the composition, in any embodiment thereof, further comprises an additional pharmaceutically active agent being contained within the composition or on a surface of the composition.

In some embodiments, the pharmaceutically active agent is selected from the group consisting of a therapeutically active agent and a labeling agent.

In some embodiments, the therapeutically active agent is selected from the group consisting of a stem cell, a growth factor, a bone morphogenetic protein, a cell, a cytokine, a hormone, a medicament, a mineral, a plasmid with therapeutic potential, and a combination thereof. As described herein, in some embodiments, the composition may further comprise at least one biologically active agent. Non-limiting biologically active agents that may be included in the composition include calcium, as well as therapeutic agents such as antibiotics, anti-inflammatory agents, growth factors, or clotting factors. Specifically, it is also possible that the disclosed composition comprises components which encourage the formation of the clot, such as, without being limited thereto, calcium salt, Factor VIII, Factor XIII, fibronectin, vitronectin, von Willebrand factor (vWF), which can be provided as a separate component or formulated with the powder.

In some embodiments, the composition is a fast soluble solid form in an aqueous medium.

The term “aqueous solution” refers to a solution comprising water as the solvent. This term may include water, saline, or a bleeding site. In some embodiments, the aqueous medium is selected from water, saline, a calcium chloride solution or a buffered aqueous medium.

In exemplary embodiments, the aqueous solution comprises saline. As used herein, the term “saline” refers to water containing salt at a concentration greater than about 0.5% and less than about 20% by weight, e.g., about 0.9 w/w % sodium chloride solution in demineralized water.

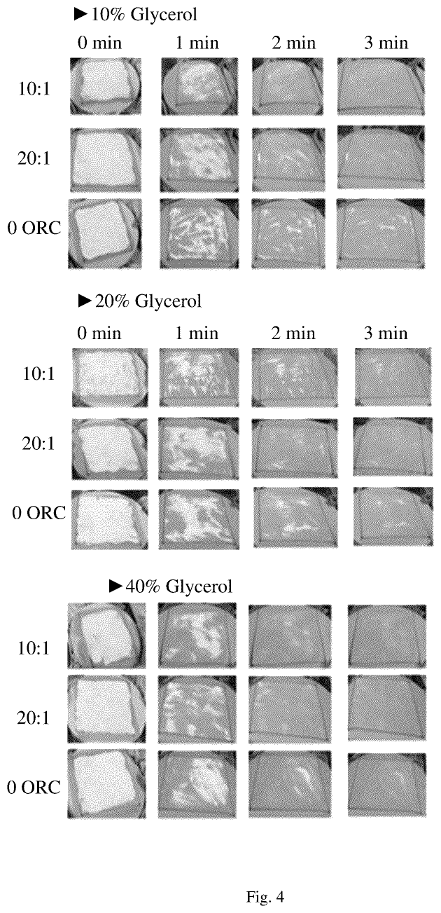

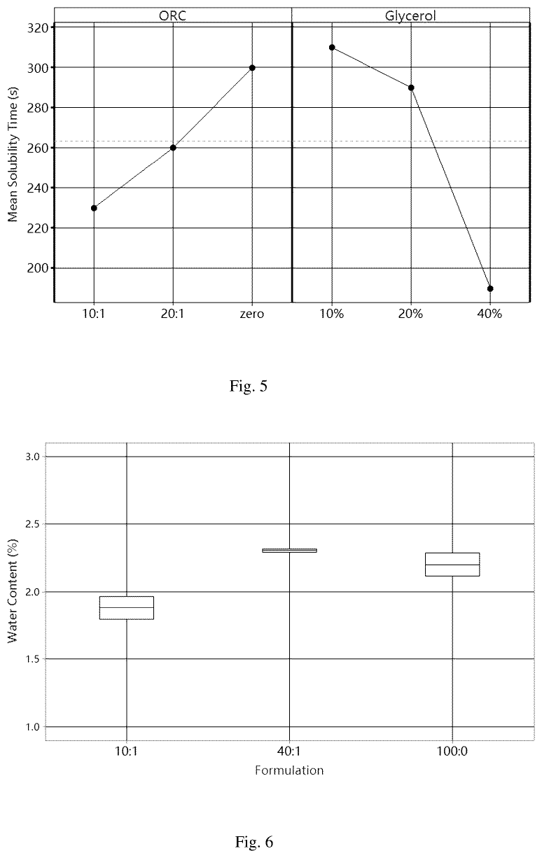

The term “fast-soluble solid” for use in the present disclosure means that the powder dissolves in less than about 350 seconds, preferably less than about 200 seconds, after placing the powder in water, see e.g., FIGS. 4 and 5.

An object of the present invention is to provide a composition which may be easily applied to a bleeding site of need, especially in difficult-to-reach areas of the body. A further advantage of the compositions of the invention is that they are bioabsorbable, and therefore may be left behind following surgery without causing any side effect.

By “applied to a bleeding site of need” it is meant to refer, e.g., to a topical application of the composition at, or on/near a bleeding surgical site of a tissue.

The composition disclosed herein may be used for any therapeutic purpose. The term “any therapeutic purpose” refers to any curative or preventive treatment in a subject. Exemplary therapeutic purposes include, but are not limited to, sealing a bore hole formed in a tissue or organ e.g., a bone; anastomosis at blood vessels; joining tissue parts e.g., soft tissue parts; treating or preventing dura defects e.g., tears and leaks following injections, fissures or cracks; treating or preventing bleeding; treating or preventing air leaks such as following pulmonary lung resection; treating or preventing defects following intestinal perforation; treating or preventing defects following anastomosis procedure carried out in any tissue e.g., uterine, esophagus, stomach, pancreas, pancreatic duct, gall bladder, bile duct, intestinal (including the small intestine and the large intestine), and rectum; treating or preventing post-operation leaks in any tissue e.g., uterine, esophagus, stomach, pancreas, pancreatic duct, gall bladder, bile duct, intestinal (including the small intestine and the large intestine), and rectum; preventing or diminishing the occurrence of post-operative leaks at the staple or suture line e.g., by applying the powdered composition according to the invention, either alone or combined with a matrix e.g., a patch, or pad onto at least a part of a defect such as a staple/suture line; for strongly affixing prosthesis e.g., during a hernia operation; for staple/suture line reinforcement; to prevent or diminish air leak, e.g., alveolar air leakage; treating or preventing renal defects; treating or preventing fistulas; treating or preventing heart defects e.g., penetrating heart wounds; reinforcing of a vascular graft prosthesis; and treating or preventing cerebrospinal fluid leakage. In some embodiments, the composition, or a matrix according to any of the embodiments disclosed herein is for use in providing hemostasis, sealing leaks and/or joining structures. In some embodiments, there is provided a method of providing hemostasis, sealing leaks and/or joining structures in a subject in need thereof, the method comprising use of the composition or the matrix according to any of the embodiments disclosed herein.

As used herein, the term “bleeding” refers to extravasation of blood from any component of the circulatory system. A “bleeding” thus encompasses unwanted, uncontrolled and often excessive bleeding in connection with surgery, trauma, or other forms of tissue damage, as well as unwanted bleedings in a patient having bleeding disorders, and severe bleeding after birth, including hemorrhage associated with caesarian section. The term “blood”, or any grammatical inflection thereof, also includes blood fractions, such as plasma.

“Wound” as used herein refers to any damage to any tissue of a patient which results in the loss of blood from the circulatory system and/or any other fluid from the patient's body. The damage may have been caused by any agent or source, including traumatic injury, infection or surgical intervention. A wound may be in a soft tissue, such as an organ, or in hard tissue, such as bone. The tissue may be an internal tissue, such as an organ or blood vessel, or an external tissue, such as the skin. The loss of blood may be internal, such as from a ruptured organ, or external, such as from a laceration.

In some embodiments, the composition, in any embodiment thereof, further comprises an additional pharmaceutically active agent being contained within the composition or on a surface of the composition.

In some embodiments, the pharmaceutically active agent is selected from the group consisting of a therapeutically active agent and a labeling agent.

In some embodiments, the therapeutically active agent is selected from the group consisting of a stem cell, a growth factor, a bone morphogenetic protein, a cell, a cytokine, a hormone, a medicament, a mineral, a plasmid with therapeutic potential, and a combination thereof. As described herein, in some embodiments, the composition may further comprise at least one biologically active agent. Non-limiting biologically active agents that may be included in the composition include calcium, as well as therapeutic agents such as antibiotics, anti-inflammatory agents, growth factors, or clotting factors. Specifically, it is also possible that the disclosed composition comprises components which encourage the formation of the clot, such as, without being limited thereto, calcium salt, Factor VIII, Factor XIII, fibronectin, vitronectin, von Willebrand factor (vWF), which can be provided as a separate component or formulated with the blend.

The pharmaceutically active agent is optionally attached to a protein (i.e. fibrinogen or fibrin) matrix (e.g., covalently linked to the protein). The agent may be attached to the matrix after the matrix has been prepared and/or attached to ingredients (e.g., protein and/or reducing sugar) which are then used to prepare the protein matrix, e.g., crosslinked fibrin.

Alternatively or additionally, the agent is absorbed by the crosslinked protein matrix. Absorption may be obtained, for example, by contacting a crosslinked fibrinogen or fibrin matrix or a non-crosslinked fibrin matrix with a solution containing the agent (e.g., by dipping, soaking, washing), or by adding the agent prior to crosslinking (e.g., inclusion of the agent in the crosslinking solution, inclusion of the agent in a solution of fibrinogen and thrombin).

Compositions described herein, as well as the contents of the abovementioned kits, may, if desired, be presented in a pack or dispenser device, which may contain one or more unit dosage forms containing the composition or ingredients (e.g., fibrinogen,) and/or reagents (e.g., an aqueous solution) for preparing the composition. The pack may, for example, comprise metal or plastic foil, such as a blister pack. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration (FDA) for prescription drugs or of an approved product insert. Compositions comprising a preparation of the invention formulated in a pharmaceutically acceptable carrier may also be prepared, placed in an appropriate container, and labeled for use for an indicated application and/or for treatment of an indicated condition, as further detailed herein. The term “preparation” refers to a physiologically suitable for therapeutic use.

It will be appreciated that compositions of embodiments of the present disclosure may be attached to or included in medical devices, such as for promoting wound healing.

In another aspect of the present disclosure, the disclosed composition in any aspect or embodiment thereof is for use in a method for preparing a fibrin sealant in/on an injured tissue of a subject, e.g., by applying the disclosed composition in any aspect and/or embodiment thereof on or in a proximity to a surface of the tissue.

In another aspect of the present disclosure, there is provided a method for preparing a fibrin sealant in/on an injured tissue of a subject, e.g., by applying the disclosed composition in any aspect and/or embodiment thereof as a hemostat on a surface of the tissue.

In another aspect of the present disclosure, there is provided a method of making the composition according to an embodiment thereof, the method comprising combining OC or ORC fibers with a fibrinogen powder and a thrombin powder in conditions allowing forming an aggregated form of the ORC fibers, fibrinogen and thrombin, upon combining the combined powders with a dispersant comprising glycerol and an aqueous solution. In another aspect of the present disclosure, there is provided a method of making the composition according to an embodiment thereof, the method comprising combining fibrinogen powder and thrombin powder in conditions allowing forming an aggregated form of the fibrinogen and thrombin, upon combining the combined powders with a dispersant comprising glycerol and an aqueous solution.

In some embodiments, such conditions allow conversion of fibrinogen into fibrin. In some embodiments, the conditions allow partial polymerization of the fibrin, e.g., by adding a certain amount of aqueous solution in the dispersant, as detailed hereinthroughout.

In some embodiments, “partial conversion” refers to 0.1% to 25% fibrinogen conversion into fibrin so as to form agglomerated particles comprising fibrinogen, thrombin, and optionally ORC, while preventing a full clotting of the fibrinogen. In some embodiments, “partial conversion” refers to 0.5% to 10% fibrinogen conversion into fibrin so as to form agglomerated particles comprising fibrinogen, thrombin, and optionally ORC, while preventing a full clotting of the fibrinogen.

Conditions that allow forming a powder/aggregated form of the ORC fibers (if present), fibrinogen and thrombin include, without being limited thereto, micronization of powders and OC fibers, e.g., by high shear process, the pH of the dispersant, e.g., 4 to 6, and temperature ranges such as, without limitation, 10 to 50° C., 45 to 50° C., or 20 to 30° C.

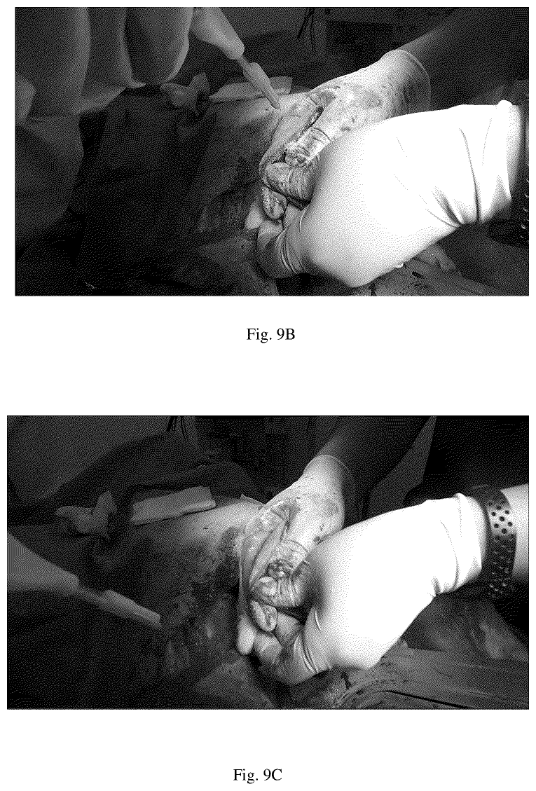

In the context of the present invention, the term “sealant”, also referred to as “fibrin sealant”, and “biological glue”, is to be understood as an adhesive, glue, or hemostat, e.g., such as one originates or being derived from the disclosed composition, having ingredients that upon contact with, or in proximity to, a tissue and/or blood, reacts to subsequently form a clot, may further act as a tissue adhesive, and thereby prevents, reduces, or stops bleeding, joins structures and/or seals physiological leaks, e.g., of cerebrospinal fluids (CSF), lymph, bile, gastrointestinal (GI) content, air leak from lungs etc. (see e.g., FIG. 8B and the Examples section that follows). In some embodiments, the sealant formulation also comprises one or more therapeutics such that upon natural degradation of the clot formed in the body, the therapeutic is released. The therapeutic may be, without being limited thereto, drug(s), such as antibiotics, analgesics, anti-inflammatory drugs, cancer drugs etc., cells including, for example, any type of stem cells e.g., Embryonic Stem (ES) cells, adult stem cells, Induced Pluripotent Stem Cells (iPSCs) etc. from human or other origin.

The term “hemostatic” refers to an ability to prevent, reduce, or stop blood loss e.g., from wounds, such as surgical or traumatic wounds, e.g., by promoting blood clot formation.

“Hemostasis” (or “haemostasis”) refers to the first stage of wound healing. It is a process which causes bleeding to stop. By “assist in hemostasis” it is meant to help reduce or stop bleeding. By “applied to a bleeding tissue” it is meant to refer to e.g., a topical application of the composition at the bleeding site, e.g., at a surgical site to control bleeding. Control of bleeding is needed in various situations including treatment of wounds.

As used herein, the terms “controlling”, “preventing”, or “reducing”, which may be used herein interchangeably in the context of the bleeding, including any grammatical inflection thereof, indicate that the rate of the blood extravagated is essentially nullified or is reduced e.g., by 10%, at least 20%, at least 30%, at least 40%, at least 50%, at least 60%, at least 70%, at least 80%, at least 90%, or even by 100%, of the initial rate of bleeding, compared to situation lacking the contact of the disclosed composition in/on the bleeding site. Methods for determining a level of appearance of bleeding are known in the art.

Further, in some embodiments, the terms “controlling”, “preventing”, or “reducing”, in the context of the bleeding are also meant to encompass at least partially sealing blood vessels at the bleeding site e.g., in soft tissues.

As used herein, the term “subject” shall mean any animal including, without limitation, a human, a mouse, a rat, a rabbit, a non-human primate, or any other mammal. In some embodiments, the subject is human, e.g., a human patient. The subject may be male or female.

As used herein, the term “injured tissue”, (or “damaged tissue”) refers to disruption of the normal continuity of structures caused by a physical (e.g., mechanical) force such as in incisions caused by surgery, a biological (e.g., thermic or actinic force), or a chemical means. The term “injured tissue” also encompasses contused tissues, as well as incised, stabbed, or lacerated tissue, as well as open, puncture, and injuries caused e.g., by ripping, scratching, pressure, and biting. The term “injured tissue” also encompasses wounds and bleeding sites as described hereinthroughout.

The term “tissue” may refer to a tissue selected from a soft tissue and a bone tissue.

The term “soft tissues” as used herein relates to body tissue that is not hardened or calcified. This term especially relates to soft tissues that are vascularized and therefore may be a source of bleeding. Examples for such tissues include, but are not limited to, connective tissue (such as tendons, ligaments, fascia, skin, fibrous tissues, fat, and synovial membranes), muscles, and internal organs. In general, soft tissues are meant to exclude bone tissue.

Thus, in some embodiments, the disclosed composition in any embodiment thereof may be used applied in difficult-to-reach bleeding sites, for example during minimally invasive surgeries (MIS) such as, e.g., endoscopy. One of the common forms of endoscopy is laparoscopy which is minimally invasive inspection and surgery inside the abdominal cavities.

The term “surgery” also encompasses the time during or after surgical or diagnostical procedures. Further non-limiting examples of procedures include neurosurgery, abdominal surgery, cardiovascular surgery, thoracic surgery, head and neck surgery, pelvic surgery and skin and subcutaneous tissue procedures. For at least one of these situations, the composition of the invention may serve as a suitable sealant, which may permit adhesion to the bleeding wound, and thus addresses hemostasis without being dependent on the condition of the wounded tissue, e.g., severity of bleeding. The formation of the sealant occurs in a relatively short period of clotting time subsequent applying the disclosed composition, in any aspect and/or embodiment thereof, on an injured tissue. Typically, using after 1 to 3 min tamponade, an incomplete clot may form which enables to stop bleeding, but more time may be needed for clotting reacting up to the paste surface thus forming a complete clot.

In another aspect of the present invention, there is provided a method of treating a wound comprising the step of applying (e.g., contacting) the disclosed composition in any aspect and/or embodiment thereof onto and/or into the wound of a subject in a need thereof.

By “treating a wound” it is further meant to encompass reducing blood loss at a bleeding site of a tissue (in vivo), e.g., in a patient undergoing surgery. Reference is made to the Examples section, e.g., FIG. 8B, showing strong adhesive properties to the porcine lung tissue for the glycerol granulated powder in this ex vivo test of tested samples disclosed herein.

Accordingly, in some embodiments, the method is for reducing blood loss at a bleeding site of a tissue and/or making a sealant layer in/on such a tissue, e.g., in a patient undergoing surgery, the method comprising contacting the disclosed composition in an embodiment thereof with the bleeding site and/or its proximity. Optionally, the method comprises first applying on such a tissue an aqueous solution, such as saline, and thereafter applying on the aqueous solution the disclosed composition, so as to promote a fast clotting-time. Accordingly, in some such embodiments, the composition, in any embodiment thereof, is for use as a hemostat in a bleeding site of a tissue.

Hereinthroughout, by “substantially devoid of” or “substantially free of” it is meant that the composition comprises less than 8%, less than 5%, less than 3%, less than 1%, or even completely absent of the relevant component (e.g., hydrophilic dispersant), for example, without limitation, low-boiling solvents. For example, the composition may be substantially devoid of polyols. In some embodiments the composition is devoid of (i.e. being less than 1%, or at times less than 0.5%, by weight) polyethylene glycol (PEG).

In some embodiments, fibrin gel made from the disclosed powder features high tensile strength. The term “tensile strength” as used herein is defined as the ultimate tensile strength of a material (e.g., the gel), which is maximum tensile force that the material can withstand without breaking. The term “high tensile strength” refers here to a tensile strength which is at least about 80 kPa, least about 90 kPa, least about 100 kPa, least about 110 kPa, least about 120 kPa, or least about 130 kPa.

In some embodiments, the fibrin gel features high tensile strength ranging from about 70 kPa to about 170 kPa, or, in some embodiments, 80 kPa to about 160 kPa, 85 kPa to about 160 kPa, as measured according to the method described in as described in PCT Application No. PCT/CN2023/082645.

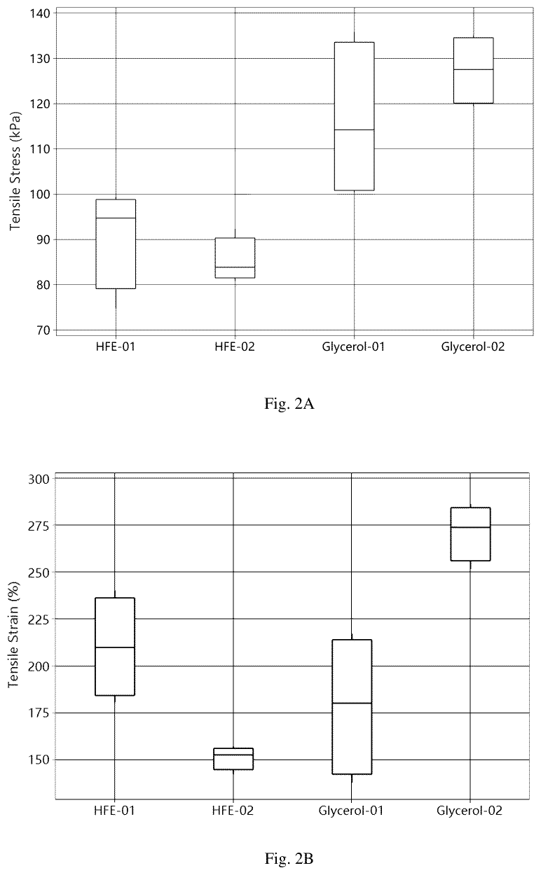

Reference is made to FIGS. 2A-2B demonstrating the stronger gel strength of fibrin gel made from the aggregated powder prepared according to embodiments of the present disclosure as compared to gels made from powders produced by hydrofluoroether (HFE).

In some embodiments, fibrin gel made from the disclosed powder features tensile strain (%) ranges from about 120% to about 190%, as measured according to the method described in as described in PCT Application No. PCT/CN2023/082645.

As used herein, the term “tensile strain” refers to the elongation of a material which is subject to tensile stress corresponding to the point of rupture.

According to an aspect of some embodiments of the present invention, there is provided a process of preparing the powder as described herein in any of the respective embodiments.

In exemplary embodiments, the general method used involves:
(a) mixing a solid fibrinogen, thrombin, and optionally OC or ORC fibers, forming a mixture;
(b) inserting the mixture in high shear mixing reactor;
(c) adding a solution of glycerol and water to the mixture in the reactor;
(d) drying and sieving the composition obtained in step (c); and thus forming the powdered hemostatic composition.

In some embodiments, the parameters of mixing in step b (also referred to as “pre-mixing”) are: impeller speed ranging from 100 to 300 rpm, chopper speed 100 to 800 rpm, and premixing for 1 to 10 min.

In some embodiments, the parameters of granulation in the high-shear reactor in step (c) (after the pre-mixing) are: impeller speed ranging from 200 to 600 rpm, chopper speed ranging from 300 to 1200 rpm, and the atomization pressure ranging from 0.005 to 0.02. Using e.g., a syringe p ump, glycerol+water solution having a glycerol-to-water ratio range of about 10% to less than about 40% by weight (e.g., 10-25 mL) is sprayed into the high-shear bowl to bind the particles, in a flowrate of e.g., 2 to 6 mL/min. Granulation step lasts e.g., 2 to 10 min, then the chopper speed changes to e.g., 800 to 1800 rpm, optionally with post granulation step lasting from e.g., 0 to 120 s. In exemplary embodiments, almost 100 g of biological powder (fibrinogen, thrombin, and optionally lysine) is combined with the 10 to 25 ml of the dispersant, with the dispersant comprising glycerol/water at certain ratios (v/v): 10% glycerol, 20% glycerol, or 40% glycerol. In exemplary embodiments, the components in in the combined powder comprises about to 80 to 96% fibrinogen powder; 300-1200 IU/dose thrombin powder; 0 to 8.77% ORC powder; 2 to 3% CaCl₂ powder; 0 to 4% lysine powder (herein % values are by weight).

In some embodiments, such conditions allow at least partial conversion of fibrinogen into fibrin. In some embodiments, the conditions allow at least partial polymerization of the fibrin, e.g., by adding a certain amount of aqueous solution in the dispersant, as detailed hereinthroughout.

In some embodiments, the mixture is substantially devoid of OC or ORC.

In some embodiments, the mixture is substantially devoid of additional polymeric binder other than fibrin.

In some embodiments, the mixture is substantially free of non-aqueous low-boiling solvent. In exemplary embodiments, the mixture is substantially free of hydrofluoroether (HFE).

As described above, in some embodiments, at least part of the powder is in the form of aggregate(s) or granulate(s). The term “granulate” or “granulate material” may particularly denote a conglomeration of particles typically below 900 micron, below 800 micron, below 700 micron, or even below 500 micron. That is, at least part of the particles is not in the form of a discrete solid. In some embodiments, high shear mix is utilized to assemble particles of fibrinogen, thrombin, and optionally CaCl₂, lysine, and ORC into aggregates.

In some embodiments, there is provided a powdered composition obtainable by the method disclosed herein in any embodiment thereof. In some embodiments, the mixture in the method comprises solid fibrinogen, thrombin, and optionally ORC fibers in solution of water and glycerol, wherein the glycerol is present in the solution (also referred to as “dispersant”) at a concentration ranging from more than about 10% to less than about 40%, by volume. In some embodiments, the glycerol is present in the solution at a concentration ranging from more than about 15% to less than about 25%, by volume. In some embodiments, the mixture is substantially devoid of OC or ORC. In some embodiments, the powdered composition is devoid of additional polymeric binder other than fibrin. In some embodiments, the mixture is substantially free of non-aqueous low-boiling solvent. In exemplary embodiments, the mixture is substantially free of hydrofluoroether (HFE). In some embodiments, the ratio of the dispersant and the powder in the granulation step is at least about 0.05 ml/g. In some embodiments, the ratio of the dispersant and the powder in the granulation step ranges from about 0.05 to about 0.35 ml/g. In some embodiments, the ratio of the dispersant and the powder ranges from about 0.1 to about 0.25 ml/g.

According to another aspect of embodiments of the present invention, a kit is provided for generating a composition described herein. It is appreciated that the consistency of the composition is such that it can be applied, for example, by spreading or by sticking the composition directly onto a damaged tissue and/or on a bleeding site. Accordingly, the composition does not need to be further spread on or applied to a solid surface, object, or other solid medium such as a strip or a film in order to be in the appropriate form for applying onto a damaged tissue or bleeding site. Nevertheless, a suitable applicator, such as, for example, a syringe, may be used in order to apply, spread or stick the composition onto the bleeding site, for the purpose of easy access and handling.

In an additional aspect and/or embodiment, the present invention provides a kit comprising: a) a container containing a composition of the invention as described above, b) an applicator for applying the composition to a tissue, and c) optionally instructions for use.

In another embodiment, the present invention further provides a hemostatic kit comprising a container containing the herein disclosed composition in an embodiment thereof.

Any aspect and embodiment of the hereinthroughout disclosed composition may be incorporated to the aspect and embodiments of the kit, including embodiments of the composition, the dispersant, and/or the powder.

Optionally, the fibrinogen and thrombin are each packaged individually (e.g., in dry form or in solution) in the kit. Thus, each of the two ingredients is packaged in separate packaging material, in addition to the packaging material of the whole kit.

Alternatively, the fibrinogen, thrombin and optionally oxidized cellulose is packaged together (e.g., as a dry powder) in the kit in the same packaging material, and optionally a dispersant (e.g., saline) is provided in an additional container. In some embodiments, the kit further comprises instructions on how to combine the ingredients of the kit and/or how to combine the ingredients of the kit with an additional ingredient (e.g., a dispersant), in order to produce the desired composition.

Optionally, the kit further comprises a dispersant. Dispersant may be e.g., water, saline or an aqueous buffer. Optionally, the dispersant is packaged individually, apart from the powder. Alternatively, the dispersant is packaged in combination with the powder, for example, as a ready-for-use composition described herein. In such embodiments, the kit may further contain a measuring means, e.g., a measuring cylinder, to measure the volume of the dispersant or a component thereof.

Additionally or alternatively, the hemostatic kit may comprise a syringe containing the blend, mixture or powder and another syringe containing the dispersant. For example, dual-syringe mixing devices may produce a substantially homogenous paste mixture by combining initially separate liquid and powders and then passing the blended contents back and forth between two connected syringes via interconnected outlet(s). Therefore, a low expression force for dispensing the paste from a syringe may be preferred for ease of mixing and ultimately for deployment of the resulting paste. The desired expression force may be less than 1.51 lbf.

In some embodiments, at least one of the containers in the kit is a pre-filled syringe. In some embodiments, a syringe is provided in addition to the container(s) of the kit. In some embodiments, the container is in a specific type, such as a vial or an applicator such as syringe.

In some embodiments, at least one of the containers in the kit is a pre-filled syringe. In some embodiments, a syringe is provided in addition to the container(s) of the kit. The term “container” may refer to any generic structure such as a vessel or a vial, that may contain the paste.

The kit may be applied using an applicator device which may be used for administering several and sequential injections of the composition. In one embodiment, the applicator device enables multiple injections of a fixed-dose of the mixed components on a 2-D surface of a tissue while moving the device. In one embodiment, the applicator has a syringe with an injection needle, which is optionally automatically retracted from the patient's skin after the injection is completed without the need for the administrator to lift the device upward from the injection surface. In one embodiment, the kit may be used for the administration of a sealant.

The hemostatic kit of the invention may be a kit for use in reducing, preventing or stopping blood flow, e.g., in open wounds, and it may be used for reducing, preventing or stopping blood flow during a procedure, such as during, before, or after a surgical procedure such as, for example, laparoscopic surgery, neurosurgery, abdominal surgery, cardiovascular surgery, thoracic surgery, head and neck surgery, pelvic surgery and skin and subcutaneous tissue procedures. The kit may be used for reducing or preventing blood flow from the skin, or in internal organs.

In some embodiments of any aspect of the kit and/or compositions, the composition is sterile. Especially when handling blood products, the sterility issue is crucial, and specifically the issue of viral inactivation. In general, viral inactivation may be carried out by any number of methods, including solvent detergent, heat inactivation, irradiation, and nanofiltration. Typically, the standard for viral inactivation requires using two different methods. Additionally, FDA standard for sterility requires filtration.

The term “sterile” as used herein means having a low bioburden, effectively being germ-free, e.g., essentially or even absolutely being free from microorganisms, e.g., bacteria and viruses. Sterilization is the process of reducing the bioburden to an effectively germ-free level. A sterile liquid or solid are generally defined as a liquid or solid that have underwent sterile filtration.

The terms “comprises”, “comprising”, “includes”, “including”, “having”, and their conjugates mean “including but not limited to”. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

In those instances where a convention analogous to “at least one of A, B, and C, etc.” is used, in general such a construction is intended in the sense one having skill in the art would understand the convention (e.g., “a composition having at least one of A, B, and C” would include but not be limited to compositions that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B”.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

The term “about” as used herein means that values that are 10% above or below the indicated value are also intended to be included. Unless otherwise indicated, all numbers such as those expressing, for example, ratios, weight, mole/mole, amounts, viscosity, temperatures, etc., are to be understood as being modified in all instances by the term “about”. Accordingly, unless indicated to the contrary, the numerical parameters set forth in this description and attached claims are approximations that may vary by up to plus or minus 10% depending upon the desired properties sought to be obtained by the present invention.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Example 1: Preparation of Micro-Agglomerate Compositions Using High-Shear Mixing (HSM) With Glycerol

### Materials and Methods:

The source of fibrinogen, thrombin and albumin was porcine blood plasma which was fractionated to obtain fibrinogen, thrombin, and albumin and supplied by Guangzhou Bioseal Biotech CO., LTD, located in Guangzhou, China; The fibrinogen and thrombin powders were produced by a spray drying process.
L-lysine-ALADDIN, L103479-500 g;
Oxidized regenerated cellulose (ORC) (if present)—the ORC powder was obtained by a milling process to the SURGICEL® original fabric;
Calcium chloride (CaCl₂), Taishan Xinning Pharmaceutical Co., Ltd.,
Glycerol, Guangzhou Chemical Reagent Factory;
Purified Water supplied by Guangzhou Bioseal Biotech Co., Ltd., Located in Guangzhou, China;
Oxidized regenerated cellulose (ORC) (if present)—characteristic: particle size<70 μm, fiber form. Briefly, the ORC powder was obtained by processing of the SURGICEL® original fabric in the following steps: 1) splitting and cutting the fabric into about 2″×8″ pieces; 2) milling the fabrics into the powder particle size (typically, but not exclusively, D50 less than 94 microns) using known milling methods; placing about 100 grams of fabric into a 500-ml zirconia jar, then placing 12 to 13 pieces of 20 mm zirconia balls (agates) into the same jar, covering and fix the jar in a Retsch planetary ball mill (Model PM100), milling the fabric with 450 rpm for 20 minutes, transferring the milled powders onto an 8″ diameter and 300—micron opening sieve, separating the agates and the powders by slightly shaking, and finally collecting the powders.

In exemplary procedures, the inventive hemostatic compositions were prepared as follows by using, inter alia, the spray drying method.

Spray drying is a drying method used to create powder from a solution, suspension or emulsion that is atomized through a spray nozzle in a hot airflow and dried instantly. Spray drying process is controlled by several process parameters, among them are: column air flow and temperatures, nozzle size and atomizing air flow rate, material flow rate, etc.

In exemplary procedures, thrombin and fibrinogen powders were produced using (each one separately) a spray drying method (Spray dryer: 4M8-TriX spray dryer was used (by ProCepT NV, Zelzate, Belgium)) as follows.

Prepared thrombin and fibrinogen porcine fibrinogen solutions were drawn into a syringe and placed inside the syringe pump of the spray dryer at various amounts. The syringe pump was set to the desired flow rate with feed valve closed. While the syringe pump feed valve was closed, the spray dryer was activated and the desired atomizing gas flow rate, drying gas flow rate, drying gas temperature, cooling gas flow rate, and cyclone gas flow rate were set. The cooling gas flow rate and temperature were selected such as not to disrupt laminar flow in the drying column, but to reduce the gas flow temperature to below the glass transition temperature of the composition in order to prevent powder from sticking to the glass parts.

The spray dryer was allowed to run until a steady state was reached where the actually measured value of the parameters reached the set levels and remained steady. The feed valve was then opened, allowing the thrombin or fibrinogen solutions to flow through the feed inlet to the spray nozzle to be atomized by the atomizing gas flow to small droplets which were then dried in the drying column. Spray dried powder was formed, and was then collected in the powder outlet of the cyclone of the spray dryer.

The spray dried powder recovered from the cyclone was weighed in a de-humidifier at a relative humidity of below 30% and divided into samples of between 100-200 mg. Each sample was individually sealed in a test tube with a plug and sealed with Parafilm® (Bemis, Oshkosh, Wisconsin, USA) until evaluation. Parameters for spraying: fan pressure 0.3 bar; atomizing pressure 3 bar; flow rate 130 ml/min; liquid pressure 16 kPa; Nozzle Diameter 0.7 mm; agitation speed 90 rpm/min.

In exemplary procedures, the powders were further granulated using high shear process.

Equipment details: Balance: SS1000; High shear mixer: Mini-CG 1 L; Syringe pump: LSPO1-1C; Vacuum dry: Vacucell 111; Vibrating screen: Fritsch analysette 3.

Environment: Temperature: 22-26° C.; Relative Humidity: 50%-70%.

The method of forming a powdered hemostatic composition comprised the steps of:

Pre-mixing: formulation which comprising fibrinogen, thrombin, oxidized regenerated cellulose (ORC) fibers, CaCl₂, and Lysine were weighted with the total amount in weight of the solids being almost 100 g. Then, materials were put into the high shear mixing reactor, the key parameters were: impeller speed 100~300 rpm, chopper speed 100~800 rpm, premix 1~10 min.

Granulation: after pre-mixing, the high shear mixing reactor parameters were changed to: impeller speed 200~600 rpm, chopper speed 300~1200 rpm, the atomization pressure was 0.005~0.02. Using a syringe pump, 10-25 mL mixed solution of glycerol and water was sprayed into the bowl to bind the particles, in a flowrate of 2~6 mL/min. In exemplary procedures, almost 100 g of biological powder (fibrinogen, thrombin, and optionally lysine) was combined with the 10 to 25 ml of the dispersant, with the dispersant comprising glycerol/water at certain ratios (v/v): 10% glycerol, 20% glycerol, or 40% glycerol. Granulation lasted 2~10 min, then the chopper speed was changed to 800~1800 rpm, post granulation 0~120 s.

Next, a first vacuum drying was applied: the obtained composition was transferred to a tray and put into vacuum drying box. The vacuum pressure was set to approximately 0 Pa for 0.5-1 hours. Next, the powder was sieved: the powder was transferred to the vibrating screen tray, the sieve size was in proper order 500/355 micron. The amplitude was set at 1.5 mm, sieving time: 10 mins. Next, a second vacuum drying was applied: the powder was transferred into the vacuum drying box, kept for drying for 3 hours, with the vacuum pressure being approximately 0 Pa. Finally, the product was collected.

Materials in the obtained powder: 80-96% fibrinogen powder; 300-1200 IU/dose thrombin powder; 0 to 8.77% ORC powder if present; 2-3% CaCl₂ powder; 0-4% lysine powder, an glycerol in the range of 0.7% to 6% (may be dependent the condition of the HSM), by weight.

A Design of Experiment (DOE) was used for formulation optimization and the following Tables provide detailed information. e.g., components and percentage, from which the corresponding powders were produced, see Tables 1A-B.

TABLE 1A
Components and Percentage

| | | | | | | | | Glycerol | |
|---|---|---|---|---|---|---|---|---|---|
| | | | | | | | | Concentration | |
| | | | | | | | Binder | in the | |
| Biological | | | | | | Total | (Fibrin) | Water | |
| Parts** | | | | | | Theoretical | Volume | Solution | Std |
| vs ORC | FIB* | THR* | CaCl₂* | ORC* | Lysine* | Input(g) | (mL) | (v/v%) | Order |
| 10:1 | 81.23 | 4.01 | 2.48 | 8.7 | 3.51 | 100 | 16 | 10% | 1 |
| 10:1 | 81.23 | 4.01 | 2.48 | 8.77 | 3.51 | 100 | 16 | 20% | 2 |
| 10:1 | 81.23 | 4.01 | 2.48 | 8.77 | 3.51 | 100 | 16 | 40% | 3 |
| 20:1 | 86.55 | 4.27 | 2.64 | 4.67 | 1.87 | 100 | 16 | 10% | 4 |
| 20:1 | 86.55 | 4.27 | 2.64 | 4.67 | 1.87 | 100 | 16 | 20% | 5 |
| 20:1 | 86.55 | 4.27 | 2.64 | 4.67 | 1.87 | 100 | 16 | 40% | 6 |
| 100:0 | 92.60 | 4.57 | 2.82 | 0.00 | 0.00 | 100 | 16 | 10% | 7 |
| 100:0 | 92.60 | 4.57 | 2.82 | 0.00 | 0.00 | 100 | 16 | 20% | 8 |
| 100:0 | 92.60 | 4.57 | 2.82 | 0.00 | 0.00 | 100 | 16 | 40% | 9 |

*In gram
** Fibrinogen + Thrombin + Lysine (w/w)

TABLE 1B
Components and Percentage

| Std Order | Run Order | Biological parts vs ORC | Glycerol |
|---|---|---|---|
| 2 | 1 | 10:1 | 20% |
| 8 | 2 | 100:0 | 20% |
| 6 | 3 | 20:1 | 40% |
| 1 | 4 | 10:1 | 10% |
| 4 | 5 | 20:1 | 10% |
| 5 | 6 | 20:1 | 20% |
| 7 | 7 | 100:0 | 10% |
| 9 | 8 | 100:0 | 40% |
| 3 | 9 | 10:1 | 40% |

### Example 2: Characterizations

The data below were generated from a DOE for formulation optimization.

### Morphology

Dry powders (see FIG. 1A) were received, uniformly blended, with a retained protein activity, controlled particle size ranges of aggregates were obtained, as shown in FIG. 1B showing scanning electron microscopy (SEM) images of exemplary aggregate. The range of the glycerol weight in the final product was 0.7-6.4 g/100 g. The size distribution of the particles in the final product: D₅₀≤about 172 mm; D₉₀≤355 mm, see Table 2; the upper limit of the ORC (the lower limit is 0%) in the final product was 8.77%.

TABLE 2
Size Distribution of a Representative Powder

| | | Run | Run | Run | Mean | |
|---|---|---|---|---|---|---|
| Sample | Size | 1(μm) | 2(μm) | 3(μm) | (μm) | RSD |
| 15-02 | D50(μm) | 171.7 | 158.2 | 157.4 | 162.43 | 4.95% |

### Tensile Stress/Stain

The obtained powder was compared to that made by hydrofluoroether (HFE) suspension (see Table 3) using a similar HSM method (as is described in U.S. patent Ser. No. 11/413,335). The comparison test results showed that the hemostatic powders made with glycerol had superior physiochemical properties and functional performance like tensile strength and tensile strain.

TABLE 3
Formulation Information - Powder Made by HFE and Glycerol Suspensions

| | | Biologicals | |
|---|---|---|---|
| Sample No. | Process | to ORC | Amount of Glycerol in the Powder |
| 21-01 | HSM -HFE | 10:1 | 0 |
| 28-01 | HSM -HFE | 10:1 | 0 |
| 10-23 | HSM - Glycerol | 10:1 | 4.8 g glycerol vs 100 g powder |
| 11-20 | HSM - Glycerol | 10:1 | 4.8 g glycerol vs 100 g powder |

### Tensile Test Procedures

In exemplary procedures, layers of powder were sprayed on an aqueous solution in a mold as described in PCT Application No. PCT/CN2023/082645. Briefly, atomized saline solution was sprayed between different layers of powder to accelerate dissolving and gelation. Upon applying the pre-weighed amount of the powder and waiting for predetermined gelling time, the gel sample was ready for testing. Next, the formed gel sample dimensions were measured and the gel was carefully clapped on to the fixture on Instron 5944 with 100N load cell. The sample was stretched, and the maximum force and corresponding displacement data were recorded electronically, and the tensile stress and tensile strain were calculated based on sample dimensions and the data collected by the instrument.

The results of the tensile stress and the tensile strength are described in Tables 4 and 5 below, respectively.

TABLE 4
Tensile Stress - Comparative Results of Gels made from Powder Produced
by HFE and Glycerol Suspensions (Four Tests for each Sample)
Tensile Stress (kPa)

| Sample 21-01 | Sample 28-01 | Sample 10-23 | Sample 11-20 |
|---|---|---|---|
| (“HFE-1”) | (“HFE-2”) | (“Glycerol-01”) | (“Glycerol-02”) |
| 92.283 | 80.89 | 126.701 | 135.100 |
| 97.142 | 83.332 | 101.74 | 132.988 |
| 74.772 | 84.408 | 135.862 | 119.444 |
| 99.371 | 92.293 | 100.498 | 122.114 |

TABLE 5
Tensile Strain - Comparative Results of Fibrin Gels made from
Powder Produced by HFE and Glycerol Suspensions
(Four Tests for each Sample)
Tensile Strain (%)

| Sample 21-01 | Sample 28-01 | Sample 10-23 | Sample 11-20 |
|---|---|---|---|
| 195.217 | 152.017 | 204.211 | 286.213 |
| 240.214 | 153.216 | 156.111 | 269.212 |
| 180.615 | 142.414 | 217.211 | 278.511 |
| 224.515 | 157.115 | 137.714 | 251.612 |

The results are further illustrated in FIGS. 2A-B, respectively.

It can be concluded that fibrin gels made from powder produced by glycerol suspensions using the HSM method are characterized by improved tensile properties as compared to the fibrin gels made from powder produced by HFE suspensions instead of glycerol.

### Effect of the ORC on Tensile Stress/Stain

In exemplary procedures, the effect of the presence of ORC on the gel's tensile stress/stain properties was examined.

The tested samples are presented in Table 6 below. Various amounts of glycerol (10%, 20%, and 40% glycerol concentrations were used were used).

TABLE 6
Formulation Information & the Effect of the Presence
of ORC on the Gel's Tensile Stress/Stain Properties

| ORC Ratio* | Glycerol** | Sample | Tensile Stress (kPa) | Tensile Strain (%) |
|---|---|---|---|---|
| 10:1 | 20% | 04-09 | 128.76 | 184.56 |
| 0 | 20% | 13-01 | 160.62 | 146.04 |
| 20:1 | 40% | 14-01 | 122.27 | 188.16 |
| 10:1 | 10% | 14-02 | 85.83 | 162.59 |
| 20:1 | 10% | 15-01 | 122.38 | 162.284 |
| 20:1 | 20% | 15-02 | 134.56 | 153.61 |
| 0 | 10% | 16-01 | 126.78 | 131.59 |
| 0 | 40% | 16-02 | 129.75 | 120.86 |
| 10:1 | 40% | 17-01 | 97.90 | 135.31 |

*ORC ratio means the ratio of biologicals vs ORC in the formulation for making the powder;
“10:1” means every 100 g biological components matches with 10 g ORC component; “20:1” means every 100 g biological components matches with 5 g ORC component; “0” means no ORC was added in the formulation.
**Relates to the glycerol concentration in the formulations from which the corresponding powders are produced.

The results are further illustrated in FIGS. 3A-B, respectively. The results are presented in mean values (for example, tensile stress data of 10:1 are mean value (104 kPa) of 128.76, 85.83, 97.90, and those data are obtained from 20%, 10%, 40% glycerol solution, respectively). It can be concluded that formulations without ORC provide powder that forms gel with the highest tensile strength, with the 20% glycerol in the formulation provide the highest tensile strength.

### Solubility Tests

In exemplary procedures, the effect of the presence of ORC on the powder's solubility was examined.

The tested samples are as presented in Table 6 above and in Table 7 below. Various amounts of glycerol (10%, 20%, and 40% glycerol concentrations were used in the preparation process).

In exemplary procedures, evenly spread 1 g powder on a 9 cm×9 cm area of a gauze which is the top of a sponge. The setting was half soaked into a saline container, the sponge was allowed to absorb saline to wet the powder, then the time until the powder totally dissolved was taken.

Referring now to FIG. 4 comparative images are shown with corresponding powders.

The comparative results are further summarized in Table 7 and are illustrated in FIGS. 5A-B.

TABLE 7
Formulation Information & the Effect of the
Presence of ORC on the Powder's Solubility

| ORC Ratio* | Glycerol** | Sample | Solubility (seconds) |
|---|---|---|---|
| 10:1 | 20% | 04-09 | 300 |
| 0 | 20% | 13-01 | 300 |
| 20:1 | 40% | 14-01 | 180 |
| 10:1 | 10% | 14-02 | 240 |
| 20:1 | 10% | 15-01 | 330 |
| 20:1 | 20% | 15-02 | 270 |
| 0 | 10% | 16-01 | 360 |
| 0 | 40% | 16-02 | 240 |
| 10:1 | 40% | 17-01 | 150 |

As found herein the invented dry powder formulations enabled the agglomerated powder to be fast dissolved in aqueous medium to form gel layer with significantly increasing gel strength and adhesive strength.

It can also be concluded that a lower amounts of ORC as well as glycerol resulted in a better solubility, however, the solubility of the powders with certain amounts of glycerol (e.g., 20%) appears to be similar even with different ratio of the ORC.

### Water Content

Water content (by weight) has significant impact on powder stability. The water content of the powder was measured by Carl Fischer's Titration method. Table 8 (see also FIG. 6) illustrates that the water content for all these batches of sample was below 2.5%.

TABLE 8
Formulation Information & the Effect of the Presence
of ORC on the Amount of Water in the Powder

| Formulation | Water content % |
|---|---|
| 10:1 | 1.7969 |
| 10:1 | 1.9648 |
| 40:1 | 2.293 |
| 40:1 | 2.3171 |
| 100:0 | 2.2862 |
| 100:0 | 2.1171 |

### Example 3: Ex Vivo and In Vivo Model Evaluations

In exemplary procedures, selected samples were tested to evaluate the hemostatic and the sealing efficacy of the powdered compositions using a various model under heparinized condition.

### Ex-Vivo Test Protocol on Porcine Lung

Fresh Porcine Lung plucks were obtained from the slaughter house. Lungs were ventilated prior to application of sample products.

In exemplary procedures, lungs were ventilated prior to application of sample products. Ventilation was initiated in a stepwise manner:
First, the ventilator was set to a Pressure Control Mode, with a breath rate of 10 bpm. Next, Expiratory Positive Airway Pressure (EPAP) was fixed to 5 cm H₂O. In exemplary procedures, Inspiratory Positive Airway Pressure (IPAP) was incrementally increased from 5-45 cm H₂O to produce a Pressure Support (PS) of 5 cm H₂O, 10 cm H₂O, 20 cm H₂O, 30 cm H₂O, and 40 cm H₂O. Next, the lung was observed to fully inflate before beginning an application of the sealant.

Next, and prior to the application of the sealant, the lungs were disconnected from the ventilator. The lungs were thereafter subject to the linear defect with a 1 cm×0.5 cm incision on the lung surface. The tested sealant was then applied to the defect while the lungs were fully deflated.

After allowing for sealants to cure, the lungs were re-connected to the ventilator, and inflated in a stepwise manner to achieve a 5 cm H₂O, 10 cm H₂O, 20 cm H₂O, 30 cm H₂O, and 40 cm H₂O.

Observations of air leak and qualitative assessment to adhere to the lung surface was recorded at each pressure step.

The results are summarized in Table 9 below.

TABLE 9
Results of Ex Vivo tests of the powders
produced from the formulations

| ORC | Glycerol | Sample | Ex Vivo Test Result |
|---|---|---|---|
| 10:1 | 20% | 04-09 | N = 2, 1^{st} 45cm no leak no bulging, 2^{nd} |
| | | | 45 cm no leak but bulging |
| 0 ORC | 20% | 13-01 | N = 1, 45 cm no leak no bulging |
| 20:1 | 40% | 14-01 | N = 1, 45 cm no leak no bulging |
| 10:1 | 10% | 14-02 | N = 1, 45 cm no leak but bulging |
| 20:1 | 10% | 15-01 | N = 2, 45 cm no leak no bulging |
| 20:1 | 20% | 15-02 | N = 1, 40 cm no leak no bulging, 45 cm |
| | | | no leak but bulging |
| 0 ORC | 10% | 16-01 | N = 1, 40 cm no leak but bulging, 45 cm |
| | | | no leak but bulging |
| 0 ORC | 40% | 16-02 | N = 1, 45 cm no leak no bulging |
| 10:1 | 40% | 17-01 | N = 1, 45 cm no leak no bulging |

It can be concluded that all tested formulations showed good sealing efficacy on ex vivo porcine lung model, with the 10:1, 400% glycerol granulated sample resulting with no leak or bulging at al.

In another set of experiments the obtained powder (made using the biologics-to-ORC 10:1 sample with the glycerol) was compared to that made by HFE suspension (10:1 sample) using the ex vivo model for the ex vivo solubility.

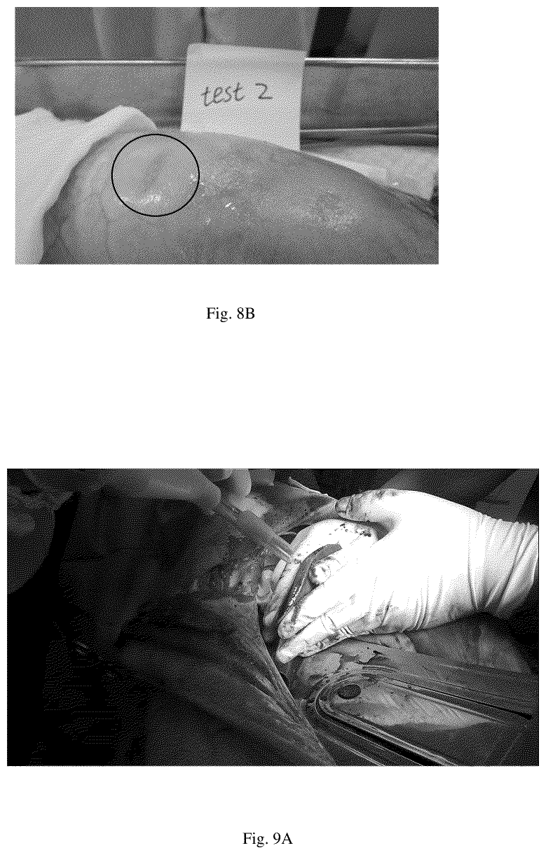

Photos were used herein to demonstrate better solubility for powder granulated with glycerol solution. As can be shown in FIG. 7 the HFE granulated sample was characterized by a poorer solubility compared to that of the glycerol granulated sample (see FIG. 81B; N=2, no air leak under 45 cm water pressure for a tested sample with 10:1 biologics to ORC with 4.8 g glycerol per 100 g powder). In addition, the HFE granulated samples (10:1 biologics to ORC) resulted in a slight bulge observed under 45 cm water pressure.

FIGS. 8A-B demonstrate strong adhesive properties to the porcine lung tissue for the glycerol granulated powder in this ex vivo test.

Conclusion: all powders made from glycerol formulations showed good sealing efficacy on ex vivo porcine lung model.

### Liver Resection Model

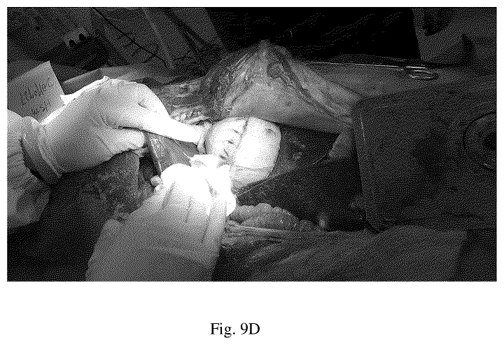

In additional exemplary procedures, liver resection model was used for powder hemostasis evaluation, as follows (representative procedure and results for the glycerol granulated powder, are shown in FIGS. 9A-9C, demonstrating hemostasis being achieved):
1. A part of the liver was resected with a scalpel or scissors (3 cm from the edge) followed by drying the surface with a gauze before powder application (see FIG. 9A).
2. Next, the sample was sprayed to cover the entire resection surface with a device, then spray an appropriate amount of saline (see FIG. 9B).
3. Step 2 can be repeated 3 to 4 times if necessary.
4. Wait 3 minutes to allow the powder to form a gel (see FIG. 9C).
5. The efficacy of immediate hemostasis was then evaluated, and the bleeding site was then rinsed with 10 mL saline and an observation was made for 1 minute to evaluate continuous hemostasis efficacy (see FIG. 9D, showing hemostasis achieved).

### Canine Lung Model

In exemplary procedures, Sealing Efficacy Testing in Canine Lung Model was performed.

### In Vivo Test Protocol on Canine Lung

The procedures were as follows:
1. Appropriate surgical procedures were used to expose lung.
2. A linear air leak defect (~L 12 mm×D 5 mm) was made in the resected lung lobe 3. The severity of the air leaks from the defect site was tested using bubble testing technique under~pressure of 20 cm H₂O.
4. The articles on were tested on their defects, then articles were allowed to cure in order to achieve optimal results.
5. The sealing efficacy was tested by inflating the lungs from a pressure 20 cm H₂O up to 30 cm H₂O to check for air bubbles.
6. The sealing efficacy was examined again at 3.5 hours post-surgery (re-open surgery site).
7. Number of animal: 7.

The results showed a sealing success rate of 100% (4/4) at time 0 and after 3.5 hours post-op for all tested samples of the glycerol granulated powder and for the ventilator pressure (cm H₂O) tested 20, 30, and 40.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A powdered composition comprising fibrinogen, thrombin, glycerol-water solution and optionally oxidized cellulose (OC), wherein:
(i) the glycerol is present at a concentration ranging from about 0.7% to about 6.5%, by weight; and wherein:
(ii) the OC is present at a concentration below about 9%, by weight, or is absent.

2. The composition of claim 1, wherein the OC comprises oxidized regenerated cellulose (ORC).

3. The composition of claim 1 or 2, being substantially free of OC and/or ORC.

4. The composition of any one of claims 1 to 3, further comprising fibrin.

5. The composition of any one of claims 1 to 4, being substantially free of hydrofluoroether.

6. The composition of any one of claims 1 to 5, being substantially free of non-aqueous low-boiling solvent.

7. The composition of any one of claims 1 to 6, having a solubility in an aqueous medium within less than 3 min at 25° C.

8. The composition of any one of claims 1 to 7, wherein the powder comprises particles characterized by a size distribution of D90 being at most 500 μm.

9. The composition of any one of claims 1 to 8, being ready-for-use for hemostatic and/or sealing indication.

10. The composition of any one of claims 1 to 9, characterized by forming a gel in an aqueous medium, said gel being characterized by a high gel strength and/or adhesive strength.

11. The composition of any one of claims 1 to 10, being substantially devoid of additional polymeric binder other than fibrin.

12. The composition of any one of claims 1 to 11, for use in a method for treating a bleeding tissue, and/or preventing an air leak in an organ in a need thereof.

13. The composition of any one of claims 1 to 12, further comprising Lysine or Tris.

14. The composition of any one of claims 1 to 13, further comprising a calcium salt.

15. The composition of any one of claims 1 to 14, being deposited on an article (e.g., a patch).

16. The composition of any one of claims 1 to 15, wherein the water is present at a concentration of up to 2.5%, by weight.

17. A method of making a powdered hemostatic composition, comprising the steps of:
(a) mixing a solid fibrinogen, thrombin, and optionally ORC fibers, forming a mixture;
(b) inserting the mixture in high shear mixing reactor;
(c) adding a solution of glycerol and water to said mixture in the reactor;
(d) drying and sieving the composition obtained in step (c), and thus forming the powdered hemostatic composition.

18. The method of claim 17, wherein the glycerol is present in said solution at a concentration ranging from more than about 10% to less than about 40% by volume.

19. The method of claim 17 or 18, wherein said water is added in a quantity preventing full clotting of fibrinogen.

20. The method of any one of claims 17 to 19, wherein the mixture is free of OC and/or ORC.

21. (canceled)

22. (canceled)

23. (canceled)

24. (canceled)

25. (canceled)

26. (canceled)

27. (canceled)

28. (canceled)

29. (canceled)

30. (canceled)
